(19) United States
(12) Patent Application Publication
(10) Pub. No.: US 2016/0038241 A1
(43) Pub. Date: Feb. 11, 2016

(54) MEDICAL PROCEDURAL ACCURACY SYSTEM

(71) Applicants: Srinivas DUTT, Land O Lakes, FL (US); Niroo DUTT, Land O Lakes, FL (US); Anisha DUTT, Land O Lakes, FL (US); Nikhil Dutt, Land O Lakes, FL (US)

(72) Inventors: Srinivas DUTT, Land O Lakes, FL (US); Niroo DUTT, Land O Lakes, FL (US); Anisha DUTT, Land O Lakes, FL (US); Nikhil Dutt, Land O Lakes, FL (US)

(21) Appl. No.: 14/815,260

(22) Filed: Jul. 31, 2015

Related U.S. Application Data
(60) Provisional application No. 62/034,315, filed on Aug. 7, 2014.

(51) Int. Cl. A61B 19/00; G09F 3/02; G09F 3/20; G09F 3/00

(52) U.S. Cl. CPC A61B 19/44; A61B 19/54; G09F 3/005; G09F 3/02; G09F 3/201; A61B 2019/446; A61B 2019/444; A61B 2019/4805; A61B 2019/5437; G09F 2003/0251

(57) ABSTRACT

A system that embodies a secure, pictorial marker to prevent surgical site errors is provided. The pictorial marker may be adapted to utilize visual processing to provide accurate and reliable information regarding a predetermined surgical site in an easy, intuitive manner. The pictorial marker may be created at the time of the decision to proceed with a surgical procedure regarding the predetermined surgical site. A systemic apparatus may protect the pictorial marker from degradation, maintain the confidentiality of the pictorial marker and other patient identifying information, and secure the pictorial marker to an appendage of the patient nearest the predetermined surgical site.

### CROSS-REFERENCE TO RELATED APPLICATION

This application claims the benefit of priority of U.S. provisional application No. 62/034,315, filed 7 Aug. 2014, the contents of which are herein incorporated by reference.

### BACKGROUND OF THE INVENTION

The present invention relates to medical procedural accuracy systems and, more particularly, to a system that embodies a secure, pictorial marker to prevent surgical site errors.

Surgery and other medical procedures are too frequently performed on the wrong site. There are multiple reasons for this and multiple techniques used to prevent it, but it continues to happen.

Markers have been used to aid surgical site accuracy, but are simple signs or written letters. These markers can be easily removed or changed. They can degrade quickly from exposure to the environment. They are also not protected from public viewing. Checklists, timeouts, written descriptions, and other techniques have been used and can be helpful but have not been sufficient in avoiding wrong site surgeries.

As can be seen, there is a need for a system that embodies a secure, pictorial marker to prevent surgical site errors by providing an easy-to-understand pictorial and anatomic description of the surgical site.

### SUMMARY OF THE INVENTION

In one aspect of the present invention, a systemic apparatus for preventing surgical errors includes a pictorial marker representing an anatomic description of a predetermined surgical site; and a privacy cover removably attached over the pictorial marker, wherein the pictorial marker is adapted to be removably attachable to a body part near the surgical site.

In another aspect of the present invention, the systemic apparatus for preventing surgical errors includes a secure band extending from a first end to a second end; a plurality of fasteners disposed near the first and second ends; a pictorial marker integrated on a portion of the secure band, wherein the pictorial marker provides a representation of an anatomic description of a predetermined surgical site; procedural information pertaining to the surgical site; and patient information pertaining to the surgical site; a transparent secure cover affixed over the pictorial marker; and a privacy cover removably attached over the pictorial marker.

In yet another aspect of the present invention, a method of preventing surgical site errors includes the steps of: providing a systemic apparatus providing a pictorial marker representing an anatomic description of a predetermined surgical site; and a privacy cover removably attached over the pictorial marker, wherein the pictorial marker is adapted to be removably attachable to a body part of a patient; making the pictorial marker when the patient and a healthcare provider have decided on a surgical procedure comprising the predetermined surgical site; removably attaching the systemic apparatus to the body part of the patient nearest the predetermined surgical site; and securing the signatures of the patient and the healthcare provider on the pictorial marker.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, shown in use;

FIG. 2 is a perspective view of an exemplary embodiment of the present invention;

FIG. 3 is an exploded view of an exemplary embodiment of the present invention;

FIG. 4 is a section view of an exemplary embodiment of the present invention, taken along line 4-4 of FIG. 1;

FIG. 5 is a top view of an exemplary embodiment of the present invention, illustrated without the privacy cover for clarity;

FIG. 6 is a schematic view of an exemplary embodiment of a pictorial marker of the present invention;

FIG. 7 is a schematic view of an exemplary embodiment of a pictorial marker of the present invention;

FIG. 8 is a schematic view of an exemplary embodiment of a pictorial marker of the present invention;

FIG. 9 is a schematic view of an exemplary embodiment of a pictorial marker of the present invention;

FIG. 10 is a schematic view of an exemplary embodiment of a pictorial marker of the present invention;

FIG. 11 is a perspective view of an exemplary embodiment of the present invention; and

FIG. 12 is an exploded view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a system that embodies a secure, pictorial marker to prevent surgical site errors. The pictorial marker may be adapted to utilize visual processing to provide accurate and reliable information regarding a predetermined surgical site in an easy, intuitive manner. The pictorial marker may be created at the time of the decision to proceed with a surgical procedure regarding the predetermined surgical site. A systemic apparatus may protect the pictorial marker from degradation, maintain the confidentiality of the pictorial marker and other patient identifying information, and secure the pictorial marker to an appendage of the patient nearest the predetermined surgical site.

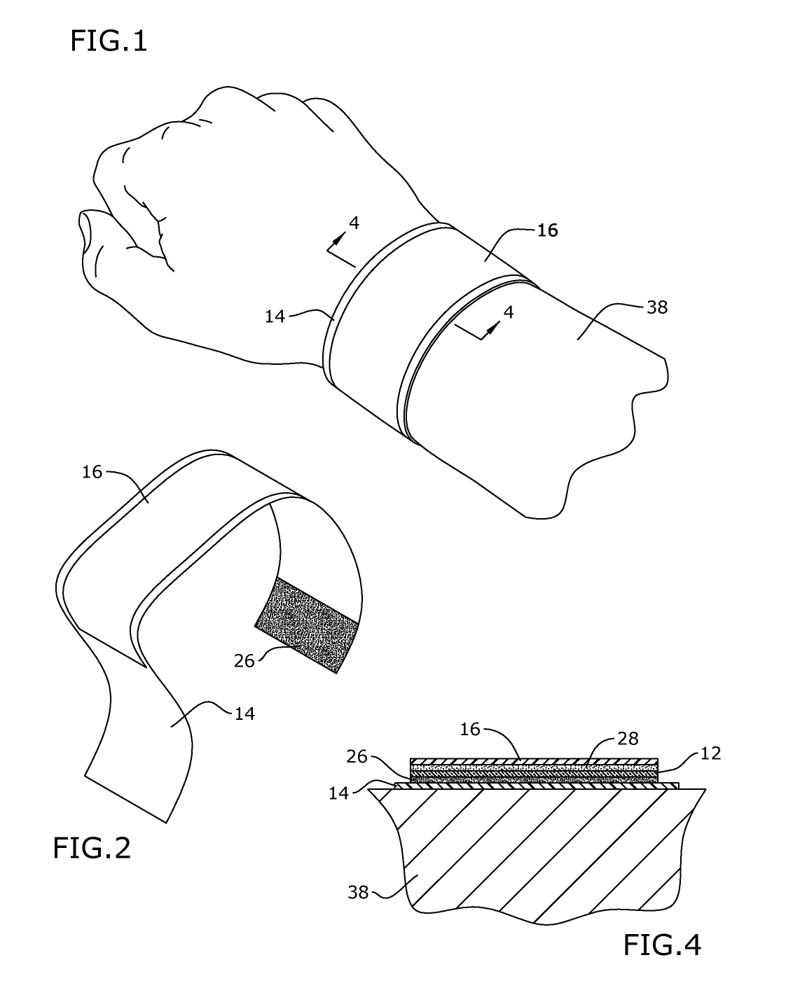

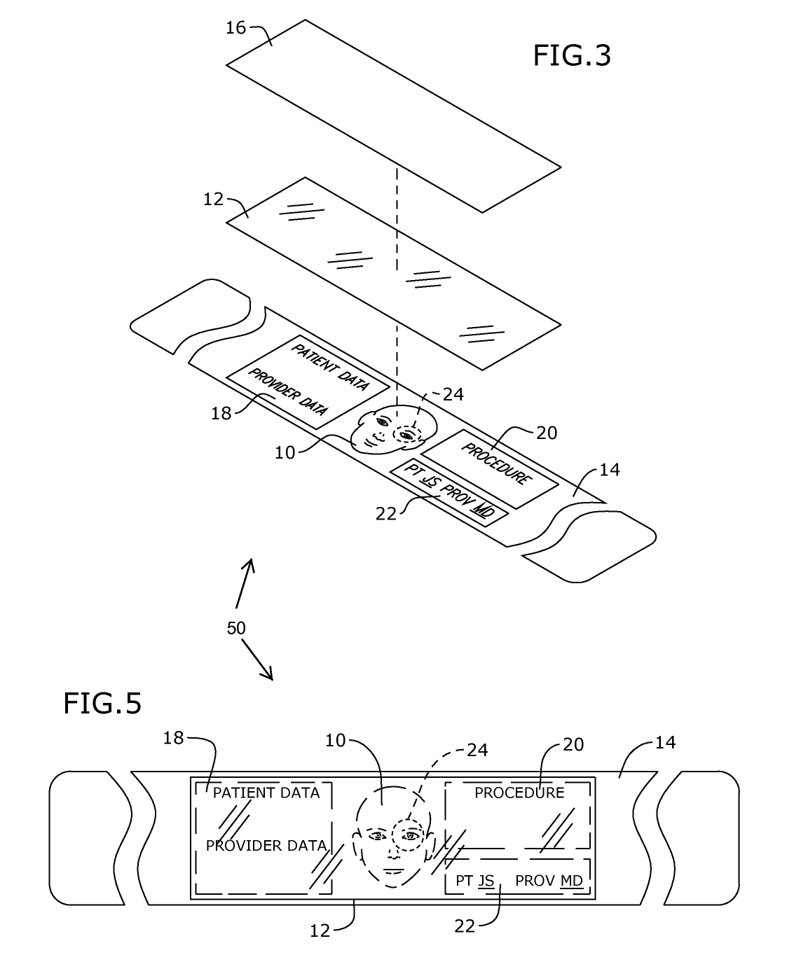

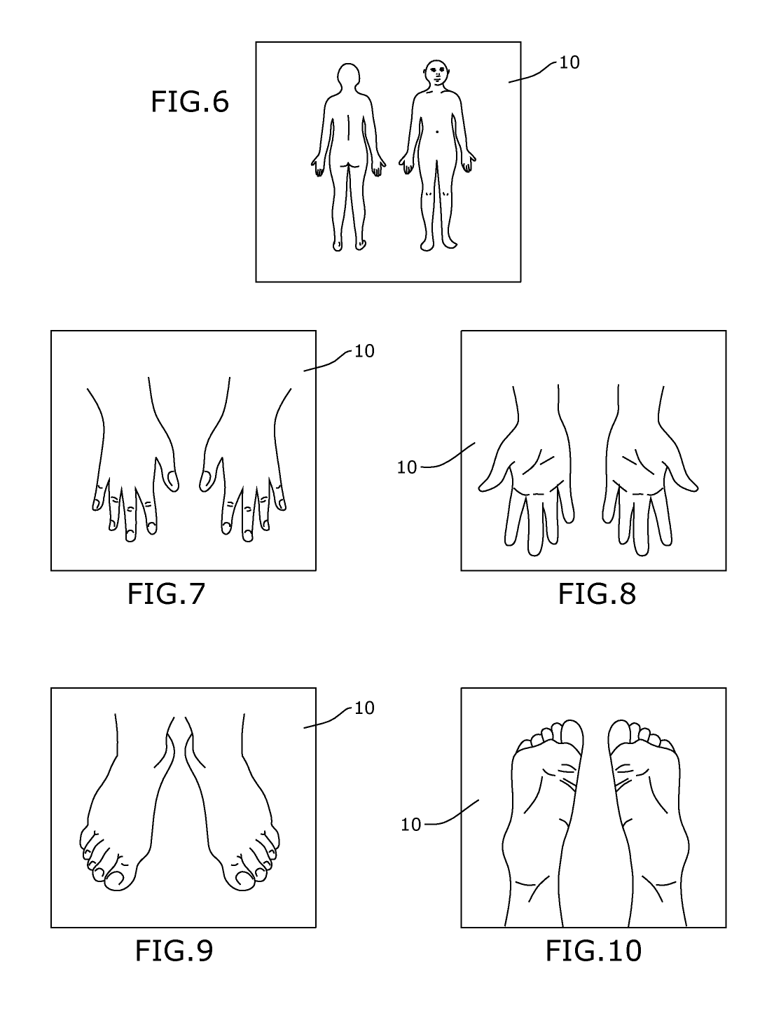

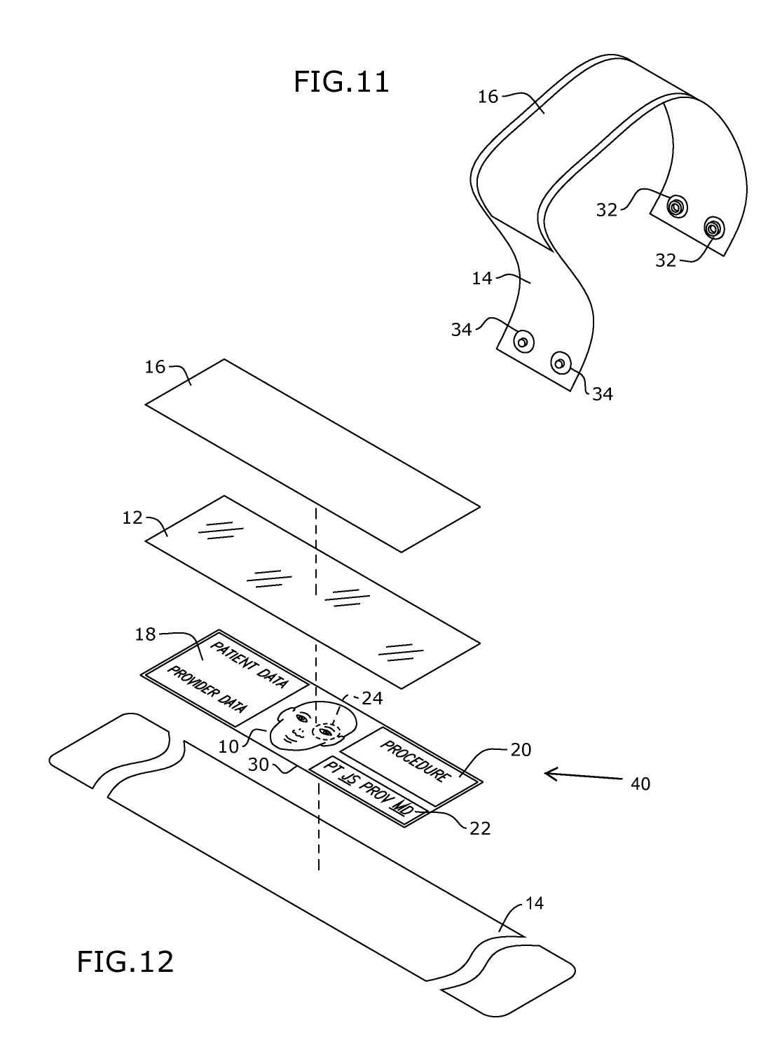

Referring to FIGS. 1 through 12, the present invention may include a system having a pictorial marker 10 embodied in a systemic apparatus 50 and method of using the same for preventing surgical site errors. The systemic apparatus 50 may include a secure band 14 made of material capable of supporting a plurality of identifying information 40 either affixed thereto or graphically printed thereon. The plurality of identifying information 40 may be incorporated on a label 30 that is affixed to the secure band 14, as illustrated in FIG. 12. Alternatively, the plurality of identifying information 40 may be printed directly on or otherwise integrally embodied in the secured band 14, as illustrated in FIG. 3. The plurality of identifying information 40 may include but not be limited to the pictorial marker 10, patient and provider data 18, procedural information 20, patient and provider approval 22, and highlighted surgical procedural area 24. In certain embodiments, the plurality of identifying information 40 may be formed with permanent ink.

The systemic apparatus 50 may include a secure cover 12 dimensioned and adapted to overlay the plurality of identifying information 40 and prevent alteration and degradation from the environment or contaminants, or otherwise further prevent the label 30 from being removed. The secure cover 12 may be made from a resilient, transparent material, such as various plasticized materials suitable for lamination. The secure cover 12 may be permanently adhered to the plurality of identifying information 40 by a sandwiched permanent adhesive layer 26, as illustrated in FIG. 4. In certain embodiments, any means of affixing the secure cover 12 to the plurality of identifying information 40 may be used.

The systemic apparatus 50 may include a privacy cover 16 dimensioned and adapted to overlay the plurality of identifying information 40 and/or secure cover 12 so as to obscure the underlying plurality of identifying information 40, preserving the confidentiality thereof. The privacy cover 16 may be made from a resilient, opaque material, such as various plasticized materials. The privacy cover 16 may be any shape, size, color, pattern and/or material that obscures the plurality of identifying information 40. The privacy cover 16 may be removably adhered to the plurality of identifying information 40 and/or secure cover 12 by a sandwiched removable adhesive layer 28, as illustrated in FIG. 4. In certain embodiments, any means of transparently removably attaching the privacy cover 14 to the plurality of identifying information 40 and/or the secure cover 12 may be used.

The secure band 14 may be dimensioned and adapted to extend from a first end to a second end so as to wrap around a body part of a predetermined user 38, whereas the first and second ends connect to secure the secure band 14 to the desired body part. A plurality of fasteners 32, 34 may be disposed near the first and second ends. The plurality of fasteners 32, 34 may be any fastener known in the art for fastening or removably securing one object to another including, for example, standard push-button snaps, Velcro-type fasteners, adhesive substances, combinations thereof, and the like. It should also be that the plurality of fasteners 32, 34 may be configured in any array and/or number, so long as the fasteners function in accordance with the present invention as described herein.

The pictorial marker 10 may embody a pictorial image visually representing a predetermined anatomic surgical site, and in some embodiments, at least a portion of the plurality of identifying information 40. The pictorial image may include any names, initials, drawings, descriptions, color, diagram, image, picture, graphical representation or the like to visually represent the predetermined surgical site, as illustrated in FIGS. 6 through 10.

The pictorial marker 10 may be adapted to utilize visual processing to provide more accurately and reliably correct site selection in an easy, intuitive manner. It compensates for human error made with written or verbal communication. Information is provided in a simple and easy to understand marked image. The pictorial marker 10 may include pre-printed templates that are partial or complete. The pictorial marker 10 may be selected on a computer or mobile device, information entered, and then printed.

A method of using the present invention may include providing the systemic apparatus 50 disclosed above. The patient 38 and surgeon may make a decision to proceed with a surgical procedure regarding the predetermined surgical site. The pictorial marker 10 may be created at this time, which is the point of clearest decision making. No confusion will occur with other patients, wrong charts, wrong dates, name misspellings, mistakes by other personnel, memory problems, rushed schedules, poor handwriting, fatigue, human errors, etc. Having the patient and physician create a secure pictorial marker together at the time of decision for a procedure, wrong site procedures can be avoided.

The pictorial marker template may be selected on a computer program, information is entered, the pictorial image may be digitally marked so as to correspond to the predetermined surgical site, and then the patient 38 and surgeon may digitally sign the pictorial marker 10. The pictorial marker 10 can be printed directly on the secure band 14 or the label 30. Alternatively, a partially pre-printed pictorial marker 10 may be manually selected. The pictorial image may be marked on the surgical site with color, signed, and finalized by both the patient and surgeon together to confirm the predetermined surgical site. Once finalized, the pictorial marker 10 may have a transparent secure cover 12 placed over it so that it cannot be modified or degraded from this point. It may be attached to the secured band 14 with the permanent adhesive 26. The opaque privacy cover 16 can then be placed over the secure cover 12 with the removable adhesive 28 to protect the confidentiality of the plurality of identifying information 40. The secure band 14 with pictorial marker 10 may be wrapped around the wrist or other appendage nearest the predetermined surgery site so that one end of the secure band 14 can be attached to its other end using the plurality of fasteners 32, 34. This prevents loss or removal of the systemic apparatus 50. A secured pictorial marker 10 of the predetermined surgery site is achieved. Healthcare personnel can remove the privacy cover 16 and compare the marked pictorial image with the actual body part. They can visually overlay the pictorial image over the patient. This visual, pictorial approach allows rapid and accurate site localization.

This systemic apparatus 50 can be used in conjunction with other techniques such as time out protocols, checklists, chart review, confirmation marks, and other techniques, providing redundancy to prevent wrong site surgeries.

Additionally, healthcare areas other than surgery could also use this systemic apparatus 50. Any procedure, imaging study, treatment, especially of bilateral sites, could use this device to improve accuracy of treatment and site.

This systemic apparatus 50 could also be used in nonsurgical areas. Technicians and repair personnel could mark areas that are in need of repair with a pictorial image and avoid mistakes and errors. An action in any field that could result in site confusion could use this pictorial device to improve accuracy and efficiency. It should be understood, of course, that the foregoing relates to exemplary embodiments of the invention and that modifications may be made without departing from the spirit and scope of the invention as set forth in the following claims.

What is claimed is:

1. A systemic apparatus for preventing surgical errors, comprising:
a pictorial marker representing an anatomic description of a predetermined surgical site; and
a privacy cover removably attached over the pictorial marker,
wherein the pictorial marker is adapted to be removably attachable to a body part near the surgical site.

2. The systemic apparatus of claim 1, wherein the pictorial marker further comprises a plurality of identifying information.

3. The systemic apparatus of claim 2, wherein the plurality of identifying information further comprises procedural information pertaining to the surgical site.

4. The systemic apparatus of claim 2, wherein the plurality of identifying information further comprises patient information pertaining to the surgical site.

5. The systemic apparatus of claim 1, further comprising a secure band integrating the pictorial marker.

6. The systemic apparatus of claim 5, wherein the secure band extends from a first end to a second end, and wherein a plurality of fasteners are disposed near the first and second ends.

7. The systemic apparatus of claim 1, further comprising a transparent secure cover affixed over the pictorial marker.

8. A systemic apparatus for preventing surgical errors, comprising:
a secure band extending from a first end to a second end;
a plurality of fasteners disposed near the first and second ends;
a pictorial marker integrated on a portion of the secure band, wherein the pictorial marker comprises:
a representation of an anatomic description of a predetermined surgical site;
procedural information pertaining to the surgical site; and
patient information pertaining to the surgical site;
a transparent secure cover affixed over the pictorial marker; and
a privacy cover removably attached over the pictorial marker.

9. A method of preventing surgical site errors, comprising the steps of:
providing a systemic apparatus comprising:
a pictorial marker representing an anatomic description of a predetermined surgical site; and
a privacy cover removably attached over the pictorial marker,
wherein the pictorial marker is adapted to be removably attachable to a body part of a patient;
making the pictorial marker when the patient and a healthcare provider have decided on a surgical procedure comprising the predetermined surgical site; and
removably attaching the systemic apparatus to the body part of the patient nearest the predetermine surgical site.

10. The method of claim 9, further comprising the step of securing the signatures of the patient and the healthcare provider on the pictorial marker.
